(19) United States
(12) Patent Application Publication
(10) Pub. No.: US 2026/0298761 A1
(43) Pub. Date: Oct. 1, 2026

(54) RAINWATER MIXED CONNECTION TRACEABILITY MONITORING DEVICE AND USING METHOD THEREFOR

(71) Applicant: THREE GORGES SMART WATER TECHNOLOGY CO., LTD, Shanghai (CN)

(72) Inventors: Mingnan CHEN, Shanghai (CN); Ruihong CHEN, Shanghai (CN); Wangchen XU, Shanghai (CN); Anwei SUN, Shanghai (CN); Jianghai CHEN, Shanghai (CN); Tong LI, Shanghai (CN)

(73) Assignee: THREE GORGES SMART WATER TECHNOLOGY CO., LTD, Shanghai (CN)

(21) Appl. No.: 19/481,155

(22) PCT Filed: Oct. 31, 2023
(86) PCT No.: PCT/CN2023/128503

§ 371 Date: Nov. 3, 2025

(30) Foreign Application Priority Data
May 25, 2023 (CN) 202310597121.8

(51) Int. Cl. G01M 3/22

(52) U.S. Cl. CPC G01M 3/22

(57) ABSTRACT

A rainwater mixed connection traceability monitoring device and a using method therefor are provided. The device includes a tracer flushing system and a tracer detection system. The tracer flushing system includes a catchment device (1) and a tracer feeding region (2). The tracer detection system includes a tracer detection device (3). The catchment device (1) includes a catchment fence a catchment plate (5), and a catchment inlet (6). The catchment plate (5) is arranged below the catchment fence The catchment inlet (6) is provided in a notch of the catchment fence The tracer feeding region (2) is arranged below one side of the catchment inlet (6). The tracer feeding region (2) includes a tracer storage region (7) and a catchment and flowing ditch (8). The catchment and flowing ditch (8) includes a flowing slot (9), a flowing partition plate (10) and a flowing bottom plate (11).

### FIELD

The present disclosure relates to the technical field of municipal monitoring and analysis, and in particular, to a rainwater mixed connection traceability monitoring device and a using method therefor.

### BACKGROUND

A drain pipeline network is a very important infrastructure in a city, and its safety and stability play an extremely important role in normal operation of the city. Due to historical reasons, a domestic pipeline network has poor overall quality at present and serious cases of mis-connection, omission, and disorderly connection, which greatly affects normal operation of the drain pipeline network and also has a huge negative impact on the quality of a water environment.

To investigate the defects in the drain pipeline network, main methods currently used include manual investigation, QV/CCTV video investigation, and the like. On the one hand, the investment cost is huge, and on the other hand, due to the lack of continuity in investigation, it can only represent a situation at the time of investigation. Meanwhile, due to the impact of pipeline waste and equipment costs, it is also hard for a traditional quality balance monitoring system based on a water volume and water quality monitoring to implement comprehensive monitoring of urban pipeline networks. There are many omissions in investigation of mixing of water (rainwater, groundwater, river water, and lake water) outside a pipeline.

Meanwhile, analysis of rainwater mixed connection is closely related to capturing of rainfall timing, and a rainfall trend in a city needs to be paid attention to all the time, and advanced monitoring preparations need to be made before raining. For a traditional analysis method for a temporary flow and a pollutant concentration, early-stage preparations waste time and labor, and there is a possibility that a delay could be caused by a false rainfall warning.

An urban pipeline network system urgently requires a convenient, low-cost, and highly sensitive rainwater mixed connection traceability monitoring device and a using method therefor.

### SUMMARY

The present disclosure aims to provide a rainwater mixed connection traceability monitoring device and a using method therefor, to solve the problems mentioned in the background section. A tracer module of the rainwater mixed connection traceability monitoring device can passively feed conductive liquids with equal concentrations and place inorganic salt particles into a sewer to monitor an environment in a tracing manner. The tracer module has a small height and effectively abuts against a curb stone, so that in a running process, the device will not affect pedestrian traffic and can be arranged for a long time, to passively capture a rainwater mixed connection working condition.

In order to achieve the above objective, the present disclosure provides the following technical solutions:

A rainwater mixed connection traceability monitoring device includes a tracer flushing system and a tracer detection system. The tracer flushing system includes a catchment device and a tracer feeding region. The tracer detection system includes a tracer detection device. The catchment device includes a catchment fence, a catchment plate, and a catchment inlet. The catchment plate is arranged below the catchment fence. The catchment inlet is provided in a notch of the catchment fence. The tracer feeding region is arranged below one side of the catchment inlet. The tracer feeding region includes a tracer storage region and a catchment and flowing ditch. The catchment and flowing ditch includes a flowing slot, a flowing partition plate, and a flowing bottom plate. The flowing bottom plate is connected to the flowing slot. The flowing partition plate is connected to the flowing slot. The tracer storage region is formed in a space of a gap between the flowing partition plate and the flowing bottom plate. A tracer flow-out slot is provided in one side of the flowing slot. A tracer mixing device is arranged at a lower end of the tracer flow-out slot. A flushing and dissolving device is arranged on one side of the tracer mixing device. The tracer detection device includes a sensor and a cover plate. The sensor is arranged at a lower end of the cover plate.

The flowing slot includes a flowing inlet and a lowering outlet; the flowing inlet is formed in a lower end of the catchment inlet; the flowing outlet and the flowing inlet are symmetrically arranged about the flowing partition plate; the tracer storage region is arranged at a lower end of the flowing inlet; a spacing is provided between the flowing partition plate and the flowing bottom plate; and the flowing partition plate partitions the flowing slot into a U-shaped water ditch.

A cloth strip can be placed on the flowing outlet and the tracer flow-out slot; and one section of the cloth strip is fixed inside the tracer flow-out slot through a fixing plate, and another section of the cloth strip is fixedly connected and arranged inside the U-shaped water ditch.

The flowing outlet is connected with the tracer flow-out slot; the tracer flow-out slot includes a flow-out slot baffle plate and a flow-out slot bottom plate; and the flow-out slot baffle plate and the flow-out slot bottom plate are respectively connected to the flowing slot at the flowing outlet.

The tracer mixing device includes a tracer mixing bottom plate; a mixture fence is arranged on a circumferential side of the tracer mixing bottom plate; the mixture fence respectively catches mixed water from the catchment device and mixed water converged along a curb stone; and the catchment device is arranged on one side of the curb stone.

An inner side of a flushing and dissolving weir body has a 90° perpendicular relationship with the tracer mixing bottom plate, and the flushing and dissolving weir body and the curb stone form an angle less than 45 degrees.

The flushing and dissolving device includes the flushing and dissolving weir body and dissolving holes. The dissolving holes are parallel to a water incoming direction. The flushing and dissolving weir body is arranged on one side of the tracer mixing device. The dissolving holes are uniformly formed in the flushing and dissolving weir body.

The dissolving holes are conical holes; and the conical holes are formed in the flushing and dissolving weir body from large to small.

The sensor includes a sensor battery and an electronic compartment; the sensor battery is connected to the electronic compartment; a probe connection port is arranged at a lower end of the sensor battery; a probe cable is arranged at a lower end of the probe connection port, a probe is arranged at a lower end of the probe cable; and the probe is an electric conductivity detection probe.

The cover plate and a lockhole of an inspection well lid are in meshing arrangement.

A tracer is one of organic salts such as sodium chloride and potassium chloride.

A using method for the rainwater mixed connection traceability monitoring device includes the following steps:
S1: the rainwater mixed connection traceability monitoring device places an inorganic salt in the tracer storage region, wherein the inorganic salt is arranged on the flowing bottom plate that is away from the lower end of the flowing partition plate by a spacing, and the inorganic salt has a thickness to ensure use in a rainwater flushing process; during raining, falling rainwater is gathered at the catchment inlet through the catchment fence and the catchment plate, and the catchment baffle plate is higher than impounded surface water, to cause collected rainwater to flow out through the catchment inlet into the flowing inlet and enter the flowing slot through the flowing inlet; the rainwater enters the flowing inlet and then falls into the tracer storage region;
S2: after the rainwater enters the tracer storage region, in case of high rainfall, the rainwater flushes and dissolves the inorganic salt, and then the rainwater contains an inorganic salt ionic solution; the inorganic salt ionic solution in the rainwater flows out through the flowing outlet into the tracer flow-out slot and enters the tracer mixing device through the tracer flow-out slot; in case of low rainfall, the cloth strip is placed on the flowing outlet and the tracer flow-out slot, and an appropriate amount of pure water is added into the tracer storage region to ensure that a lower edge of the cloth strip to be slightly higher than a water level of added water; the inorganic salt ionic solution in the rainwater flows out under a capillary action of the cloth strip, enters the tracer flow-out slot, and enters the tracer mixing device through the tracer flow-out slot;
S3: the inorganic salt ionic solution in the tracer mixing device flows out through the tracer flow-out slot, and flows into the tracer mixing device in the flow-out process, other rainwater is converged towards a road curb through a ground and enters the tracer mixing bottom plate along a curb stone for mixing; in the process, mixed rainwater enters the flushing and dissolving device; the flushing and dissolving weir body of the flushing and dissolving device is higher than the tracer mixing bottom plate; in a flowing process, a water stratification layer is formed; inorganic salt particles brought out by the rainwater of the water stratification layer are dissolved and flushed in the dissolving holes in the flushing and dissolving weir body to prevent the inorganic salt particles from flowing out and causing inaccurate testing; after the inorganic salt is dissolved in the dissolving holes, the mixed rainwater enters an urban pipeline; and
S4: after the rainwater enters the urban pipeline, a plurality of tracer detection devices are arranged in lockholes of different inspection well lids in the urban pipeline to ensure monitoring of flowing out of inorganic salt rainwater; rainwater traceability monitoring is performed through the sensors of the tracer detection devices; in a monitoring process, by monitoring electric conductivity by the probes, in-water detection of the probes and monitoring of traceability substances in water are implemented; qualitative monitoring of mixed connection and semi-quantitative analysis of a mixed connection distance are implemented based on a peak value of batch conductivity variation,

Compared with the prior art, the present disclosure has the beneficial effects below:
1) A tracer module of the rainwater mixed connection traceability monitoring device can passively feed conductive liquids with equal concentrations and place inorganic salt particles into a sewer to monitor an environment in a tracing manner.
2) The catchment device is arranged to effectively abut against the curb stone, so that the device cannot affect the pedestrian traffic in a running process.
3) The tracer cannot be affected by environments and can be placed at a location to be tested for a long time period before raining. It is convenient to prepare the tracer.
4) An outlet of the tracer module is in an elastic design, so that batch releasing of the tracer is implemented to provide more information for traceability analysis.
5) The cloth strip is arranged at the outlet of the tracer module. With a capillary phenomenon, the tracer can be still released in a case of insufficient rainfall.
6) The inner side of the flushing and dissolving weir body bas a 90° perpendicular relationship with the tracer mixing bottom plate, and the flushing and dissolving weir body and the curb stone form an angle less than 45 degrees. Under the guide action of the weir body, water is gathered below the outlet of the tracer module. Meanwhile, since the inner side of the flushing and dissolving weir body is perpendicular to a barrier wall, a strong mixed vortex is formed below the outlet of the tracer module to accelerate tracer mixing.
7) a through hole is formed in a lower part of the weir body, which can guide fine sand and prevent the weir body from being blocked to weaken mixing.
8) An electric conductivity monitoring module of the rainwater mixed connection traceability monitoring device can simultaneously implement detection by putting the probe into water and monitor traceability substances in water.
9) The electric conductivity monitoring module of the rainwater mixed connection traceability monitoring device can adapt to the lockhole of the inspection well lid to implement mounting without opening a cover.
10) A gap exists between the electric conductivity monitoring module of the rainwater mixed connection traceability monitoring device and the lockhole of the inspection well lid, and the probe can be effectively shook by being rolled by a wheel, to prevent sludge from being stuck onto the probe.
11) In a rainwater mixed connection traceability monitoring analysis method, harmless, inexpensive, and inorganic substances such as sodium chloride are used as tracers. By virtue of a tracer feeding module and a tracer monitoring module, qualitative monitoring of mixed connection and semi-quantitative analysis of a mixed connection distance are implemented based on a peak value of batch conductivity variation.

### BRIEF DESCRIPTION OF THE DRAWINGS

FIG. 1 is a schematic diagram of a structural principle of a rainwater mixed connection traceability monitoring device according to the present disclosure.

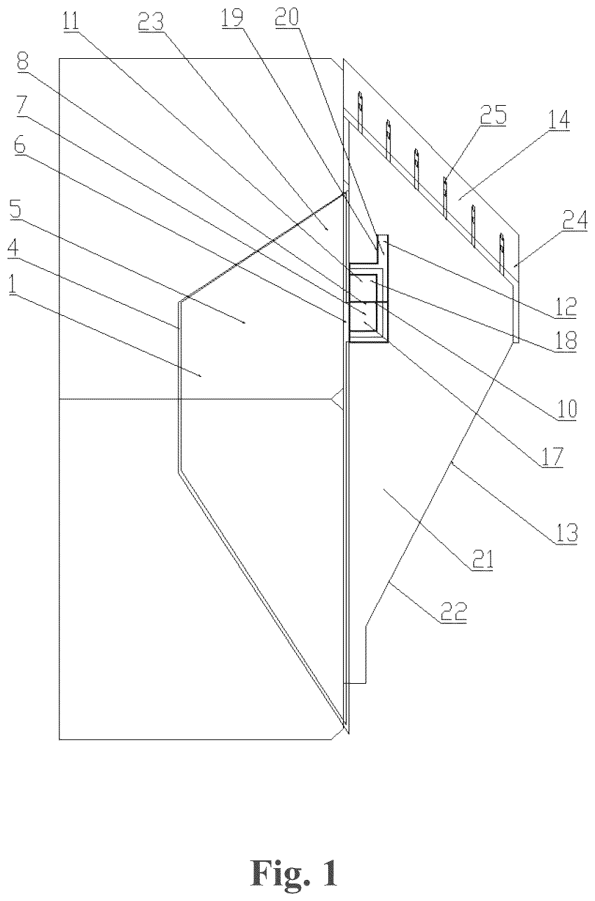

FIG. 2 is a schematic diagram of a side-view structure of a rainwater mixed connection traceability monitoring device according to the present disclosure.

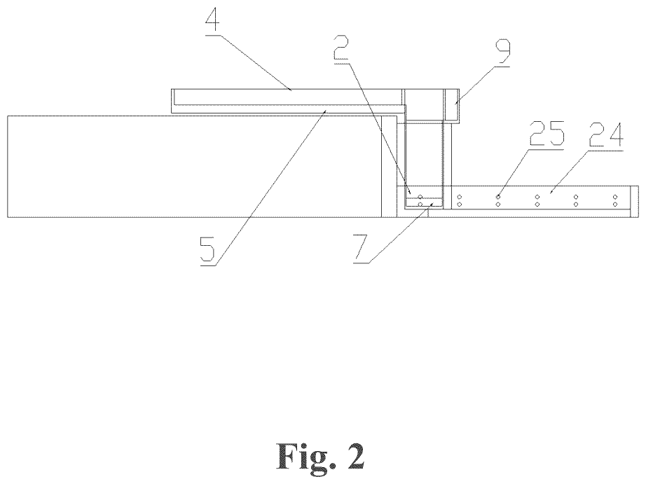

FIG. 3 is a schematic diagram of a structural principle of a catchment device according to the present disclosure,

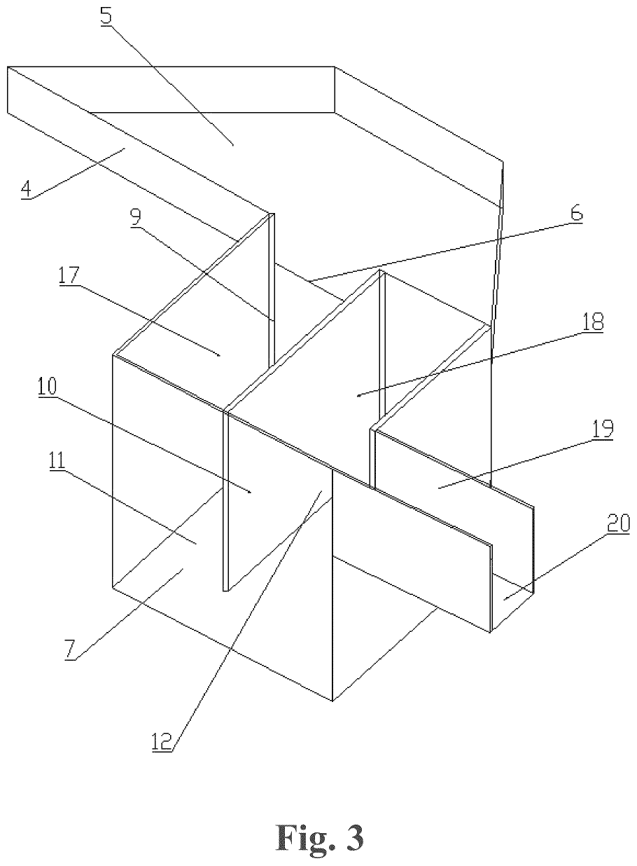

FIG. 4 is a schematic diagram of a structural principle of a flushing and dissolving device according to the present disclosure.

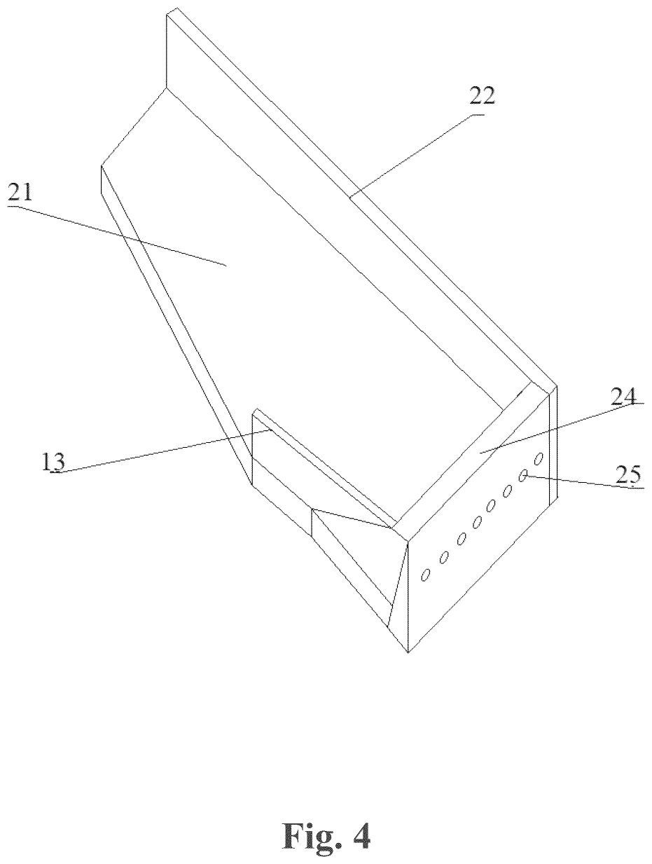

FIG. 5 is a schematic diagram of a structural principle of a tracer detection device according to the present disclosure.

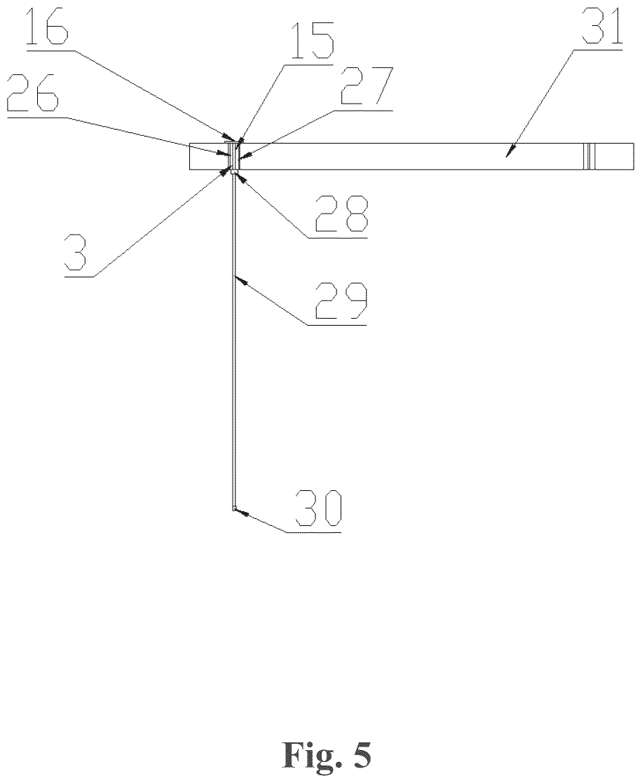

FIG. 6 is a schematic structural diagram of combination of a cover plate and an inspection well lid according to the present disclosure.

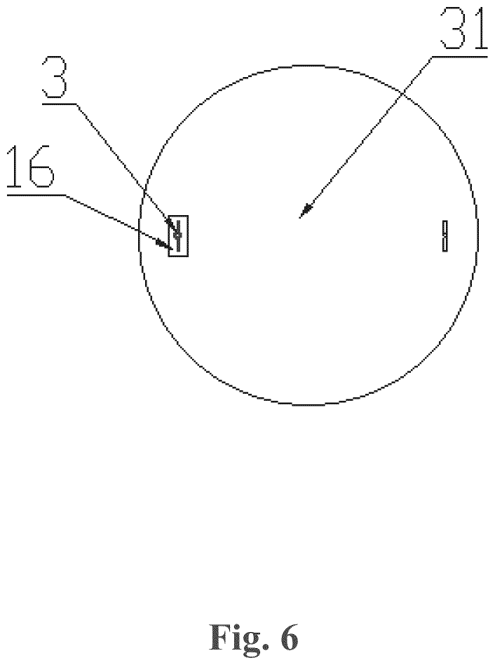

FIG. 7 is a schematic diagram of a structural principle of a position of a cloth strip according to the present disclosure.

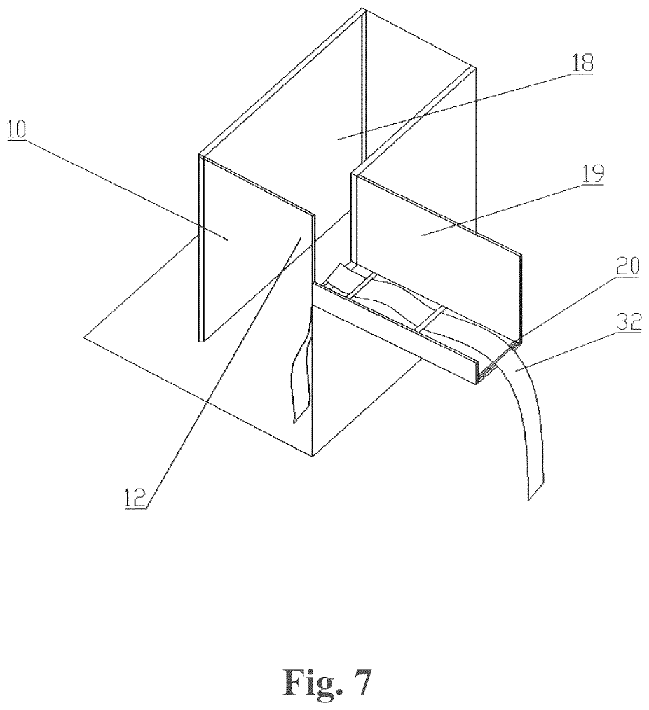

Reference numerals in the accompanying drawings: 1: catchment device; 2: tracer feeding region; 3: tracer detection device; 4: catchment fence; 5: catchment plate; 6: catchment inlet; 7: tracer storage region, 8: catchment and flowing ditch; 9: flowing slot; 10: flowing partition plate; 11: flowing bottom plate; 12: tracer flow-out slot; 13: tracer mixing device; 14: flushing and dissolving device, 15: sensor; 16: cover plate; 17: flowing inlet; 18: flowing outlet; 19: flow-out slot baffle plate; 20: flow-out slot bottom plate; 21: tracer mixing bottom plate; 22: mixture fence; 23: curb stone; 24: flushing and dissolving weir body; 25: dissolving hole, 26: sensor battery; 27: electronic compartment; 28: probe connection port; 29: probe cable; 30: probe; 31: inspection well lid; and 32: cloth strip.

### DETAILED DESCRIPTION

Specific implementations of the present disclosure are described in detail based on the following content with reference to the accompanying drawings.

A rainwater mixed connection traceability monitoring device includes a tracer flushing system and a tracer detection system. The tracer flushing system includes a catchment device 1 and a tracer feeding region 2. The tracer detection system includes a tracer detection device 3. The catchment device 1 includes a catchment fence 4, a catchment plate 5, and a catchment inlet 6. The catchment plate 5 is arranged below the catchment fence 4. The catchment inlet 6 is provided in a notch of the catchment fence 4. The tracer feeding region 2 is arranged below one side of the catchment inlet 6. The tracer feeding region 2 includes a tracer storage region 7 and a catchment and flowing ditch 8. The catchment and flowing ditch 8 includes a flowing slot 9, a flowing partition plate 10, and a flowing bottom plate 11. The flowing bottom plate 11 is connected to the flowing slot 8. The flowing partition plate 10 is connected to the flowing slot 8. The tracer storage region 7 is formed in a space of a gap between the flowing partition plate 10 and the flowing bottom plate 11. A tracer flow-out slot 12 is provided in one side of the flowing slot 8. A tracer mixing device 13 is arranged at a lower end of the tracer flow-out slot 12 A flushing and dissolving device 14 is arranged on one side of the tracer mixing device 13. The tracer detection device 3 includes a sensor 15 and a cover plate 16. The sensor 15 is arranged at a lower end of the cover plate 16. A tracer module of the rainwater mixed connection traceability monitoring device can passively feed conductive liquids with equal concentrations and place inorganic salt particles into a sewer to monitor an environment in a tracing manner. The tracer module has a small height and effectively abuts against a curb stone, so that in a running process, the device will not affect pedestrian traffic. An outlet of the tracer module is in an elastic design, so that batch releasing of the tracer is implemented to provide more information for traceability analysis.

The flowing slot 8 includes a flowing inlet 17 and a lowering outlet 18. The flowing inlet 17 is formed in a lower end of the catchment inlet 6. The flowing outlet 18 and the flowing inlet 17 are symmetrically arranged about the flowing partition plate 10. The tracer storage region 7 is arranged at a lower end of the flowing inlet 17. A spacing is provided between the flowing partition plate 10 and the flowing bottom plate 11. The flowing partition plate 10 partitions the flowing slot 8 into a U-shaped water ditch.

The flowing outlet 18 is connected with the tracer flow-out slot 12. The tracer flow-out slot 12 includes a flow-out slot baffle plate 19 and a flow-out slot bottom plate 20. The flow-out slot baffle plate 19 and the flow-out slot bottom plate 20 are respectively connected to the flowing slot 8 at the flowing outlet 18.

A cloth strip 32 can be placed on the flowing outlet 18 and the tracer flow-out slot 12; and one section of the cloth strip 32 is fixed inside the tracer flow-out slot 12 through a fixing plate, and another section of the cloth strip 32 is fixedly connected and arranged inside the U-shaped water ditch.

The tracer mixing device 13 includes a tracer mixing bottom plate 21, a mixture fence 22 is arranged on a circumferential side of the tracer mixing bottom plate 21; the mixture fence 22 respectively catches mixed water from the catchment device I and mixed water that is converged towards a road curb through a ground along a curb stone 23. The catchment device 1 is arranged on one side of the curb stone 23.

The flushing and dissolving device 14 includes a flushing and dissolving weir body 24 and dissolving holes 25. The flushing and dissolving weir body 24 is arranged on one side of the tracer mixing device 13. The dissolving holes 25 are uniformly formed in the flushing and dissolving weir body 24.

The dissolving holes 25 are conical holes; and the conical holes are formed in the flushing and dissolving weir body from large to small.

The sensor 15 includes a sensor battery 26 and an electronic compartment 27. The sensor battery 26 is connected to the electronic compartment 27. A probe connection port 28 is arranged at a lower end of the sensor battery 26. A probe cable 29 is arranged at a lower end of the probe connection port 28. A probe 30 is arranged at a lower end of the probe cable 29. The probe 30 is an electric conductivity detection probe. An electric conductivity monitoring module of the rainwater mixed connection traceability monitoring device can simultaneously implement detection by putting the probe into water and monitor traceability substances in water.

The cover plate 16 and a lockhole of an inspection well lid 31 are in meshing arrangement. The electric conductivity monitoring module of the rainwater mixed connection traceability monitoring device can adapt to the lockhole of the inspection well lid to implement mounting without opening a cover. A gap exists between the electric conductivity monitoring module and the lockhole of the inspection well lid, and the monitoring probe can be effectively shook by being rolled by a wheel, to prevent sludge from being stuck onto the probe.

A tracer is one of organic salts such as sodium chloride and potassium chloride. In a rainwater mixed connection traceability monitoring analysis method, harmless, inexpensive, and inorganic substances such as sodium chloride are used as tracers. By virtue of a tracer feeding module and a tracer monitoring module, qualitative monitoring of mixed connection and semi-quantitative analysis of a mixed connection distance are implemented based on a peak value of batch conductivity variation.

A using method for the rainwater mixed connection traceability monitoring device in case of heavy rain includes the following steps:
S1: the rainwater mixed connection traceability monitoring device places an inorganic salt in the tracer storage region 7, where the inorganic salt is arranged on the flowing bottom plate 11 that is away from the lower end of the flowing partition plate 10 by a spacing, and the inorganic salt has a thickness to ensure use in a rainwater flushing process; during raining, falling rainwater is gathered at the catchment inlet 6 through the catchment fence 4 and the catchment plate 5, and the catchment baffle plate 4 is higher than impounded surface water, to cause collected rainwater to flow out through the catchment inlet 6 into the flowing inlet 17 and enter the flowing slot 9 through the flowing inlet 17; the rainwater enters the flowing inlet 17 and then falls into the tracer storage region 7;
S2: after entering the tracer storage region 7, the rainwater flushes and dissolves the inorganic salt, and then the rainwater contains an inorganic salt ionic solution; the inorganic salt ionic solution in the rainwater flows out through the flowing outlet 18 into the tracer flow-out slot 12 and enters the tracer mixing device 13 through the tracer flow-out slot 12;
S3: the inorganic salt ionic solution in the tracer mixing device 13 flows out through the tracer flow-out slot 12, and flows into the tracer mixing device 13 in the flow-out process; other rainwater is converged towards road curbs on two sides through a road and enters the tracer mixing bottom plate 21 along the curb stone 23 for mixing; in this process, mixed rainwater enters the flushing and dissolving device 14; the flushing and dissolving weir body 24 of the flushing and dissolving device 14 is higher than the tracer mixing bottom plate 21; in a flowing process, a water stratification layer is formed, which accelerates inorganic salt mixing; after the inorganic salt is dissolved, the inorganic salt is mixed with the rainwater and enters an urban pipeline; and
S4: after the rainwater enters the urban pipeline, a plurality of tracer detection devices 3 are arranged in lockholes of different inspection well lids of the urban pipeline to ensure monitoring of flowing out of inorganic salt rainwater; rainwater traceability monitoring is performed through the sensors of the tracer detection devices 3; in a monitoring process, by monitoring electric conductivity by the probes, in-water detection of the probes and monitoring of traceability substances in water are implemented; qualitative monitoring of mixed connection and semi-quantitative analysis of a mixed connection distance are implemented based on a peak value of batch conductivity variation.

A using method for the rainwater mixed connection traceability monitoring device in case of light rain includes the following steps:
S1: the rainwater mixed connection traceability monitoring device places an inorganic salt in the tracer storage region 7, where the inorganic salt is arranged on the flowing bottom plate 11 that is away from the lower end of the flowing partition plate 10 by a spacing, and the inorganic salt has a thickness to ensure use in a rainwater flushing process;
the cloth strip 32 is placed on the flowing outlet 18 and the tracer flow-out slot 12; and an appropriate amount of pure water is added into the tracer storage region 7 to ensure that a lower edge of the cloth strip 32 to be slightly higher than a water level of added water;
during raining, falling rainwater is gathered at the catchment inlet 6 through the catchment fence 4 and the catchment plate 5, and the catchment fence 4 is higher than impounded surface water, to cause collected rainwater to flow out through the catchment inlet 6 into the flowing inlet 17 and enter the flowing slot 9 through the flowing inlet 17; the rainwater enters the flowing inlet 17 and then falls into the tracer storage region 7;
S2: after entering the tracer storage region 7, the rainwater flushes and dissolves the inorganic salt, and then the rainwater contains an inorganic salt ionic solution;
a water volume is limited; the inorganic salt ionic solution in the rainwater Dows out under a capillary action of the cloth strip 32, enters the tracer flow-out slot 12, and enters the tracer mixing device 13 through the tracer flow-out slot 12;
S3: the inorganic salt ionic solution in the tracer mixing device 13 flows out through the tracer flow-out slot 12, and flows into the tracer mixing device 13 in the flow-out process; other rainwater is converged towards road curbs on two sides through a road and enters the tracer mixing bottom plate 21 along the curb stone 23 for mixing; in this process, mixed rainwater enters the flushing and dissolving device 14; the flushing and dissolving weir body 24 of the flushing and dissolving device 14 is higher than the tracer mixing bottom plate 21; in a flowing process, a water stratification layer is formed, which accelerates inorganic salt mixing; after the inorganic salt is dissolved, the inorganic salt is mixed with the rainwater and enters an urban pipeline; and
S4: after the rainwater enters the urban pipeline, a plurality of tracer detection devices 3 are arranged in lockholes of different inspection well lids of the urban pipeline to ensure monitoring of flowing out of inorganic salt rainwater; rainwater traceability monitoring is performed through the sensors of the tracer detection devices 3; in a monitoring process, by monitoring electric conductivity by the probes, in-water detection of the probes and monitoring of traceability substances in water are implemented; qualitative monitoring of mixed connection and semi-quantitative analysis of a mixed connection distance are implemented based on a peak value of batch conductivity variation.

The foregoing descriptions are preferable implementations of the present disclosure only. It is noted that a person skilled in the art may make some improvements and replacements without departing from the technical principle of the present disclosure and the improvements and replacements shall fall within the protection scope of the present disclosure.

What is claimed is:

1. A rainwater mixed connection traceability monitoring device, comprising a tracer flushing system and a tracer detection system,
wherein the tracer flushing system comprises a catchment device and a tracer feeding region, the tracer detection system comprises a tracer detection device, the catchment device comprises a catchment fence, a catchment plate, and a catchment inlet; the catchment plate is arranged below the catchment fence; the catchment inlet is provided in a notch of the catchment fence; the tracer feeding region is arranged below one side of the catchment inlet;
the tracer feeding region comprises a tracer storage region and a catchment and flowing ditch; the catchment and flowing ditch comprises a flowing slot, a flowing partition plate, and a flowing bottom plate; the flowing bottom plate is connected to the flowing slot; the flowing partition plate is connected to the flowing slot; the tracer storage region is formed in a space of a gap between the flowing partition plate and the flowing bottom plate; a tracer flow-out slot is provided in one side of the flowing slot;
a tracer mixing device is arranged at a lower end of the tracer flow-out slot; a flushing and dissolving device is arranged on one side of the tracer mixing device; the tracer detection device comprises a sensor and a cover plate; and the sensor is arranged at a lower end of the cover plate.

2. The rainwater mixed connection traceability monitoring device according to claim 1, wherein the flowing slot comprises a flowing inlet and a lowering outlet; the flowing inlet is formed in a lower end of the catchment inlet; the flowing outlet and the flowing inlet are symmetrically arranged about the flowing partition plate; the tracer storage region is arranged at a lower end of the flowing inlet; a spacing is provided between the flowing partition plate and the flowing bottom plate; and the flowing partition plate partitions the flowing slot into a U-shaped water ditch.

3. The rainwater mixed connection traceability monitoring device according to claim 2, wherein the flowing outlet is connected with the tracer flow-out slot; the tracer flow-out slot comprises a flow-out slot baffle plate and a flow-out slot bottom plate; and the flow-out slot baffle plate and the flow-out slot bottom plate are respectively connected to the flowing slot at the flowing outlet.

4. The rainwater mixed connection traceability monitoring device according to claim 1, wherein the tracer mixing device comprises a tracer mixing bottom plate; a mixture fence is arranged on a circumferential side of the tracer mixing bottom plate; the mixture fence respectively catches mixed water from the catchment device and mixed water that is converged towards a road curb through a ground along a curb stone; and the catchment device is arranged on one side of the curb stone.

5. The rainwater mixed connection traceability monitoring device according to claim 1, wherein the flushing and dissolving device comprises a flushing and dissolving weir body and dissolving holes; the flushing and dissolving weir body is arranged on one side of the tracer mixing device; the dissolving holes are uniformly formed in the flushing and dissolving weir body; the dissolving holes are conical holes, and the conical holes are formed in the flushing and dissolving weir body from large to small.

6. The rainwater mixed connection traceability monitoring device according to claim 2, wherein a cloth strip is placed on the flowing outlet and the tracer flow-out slot; and one section of the cloth strip is fixed inside the tracer flow-out slot through a fixing plate, and another section of the cloth strip is fixedly connected and arranged inside the U-shaped water ditch.

7. The rainwater mixed connection traceability monitoring device according to claim 1, wherein the sensor comprises a sensor battery and an electronic compartment, the sensor battery is connected to the electronic compartment; a probe connection port is arranged at a lower end of the sensor battery, a probe cable is arranged at a lower end of the probe connection port; a probe is arranged at a lower end of the probe cable, and the probe is an electric conductivity detection probe.

8. The rainwater mixed connection traceability monitoring device according to claim 1, wherein the cover plate and a lockhole of an inspection well lid are in meshing arrangement.

9. The rainwater mixed connection traceability monitoring device according to claim 7, wherein a tracer is one of organic salts such as sodium chloride and potassium chloride.

10. A using method for a rainwater mixed connection traceability monitoring device, wherein the rainwater mixed connection traceability monitoring device is the rainwater mixed connection traceability monitoring device according to claim 1, and the using method comprises the following steps:
S1: the rainwater mixed connection traceability monitoring device places an inorganic salt in the tracer storage region, wherein the inorganic salt is arranged on the flowing bottom plate that is away from the lower end of the flowing partition plate by a spacing, and the inorganic salt has a thickness to ensure use in a rainwater flushing process; during raining, falling rainwater is gathered at the catchment inlet through the catchment fence and the catchment plate, and the catchment baffle plate is higher than impounded surface water, to cause collected rainwater to flow out through the catchment inlet into the flowing inlet and enter the flowing slot through the flowing inlet; the rainwater enters the flowing inlet and then falls into the tracer storage region;
S2: after the rainwater enters the tracer storage region, in case of high rainfall, the rainwater flushes and dissolves the inorganic salt, and then the rainwater contains inorganic salt solution ions; the inorganic salt ionic solution in the rainwater flows out through the flowing outlet into the tracer flow-out slot and enters the tracer mixing device through the tracer flow-out slot; in case of low rainfall, the cloth strip is placed on the flowing outlet and the tracer flow-out slot, and an appropriate amount of pure water is added into the tracer storage region to ensure that a lower edge of the cloth strip to be slightly higher than a water level of added water, the inorganic salt ionic solution in the rainwater flows out under a capillary action of the cloth strip, enters the tracer flow-out slot, and enters the tracer mixing device through the tracer flow-out slot;
S3: the inorganic salt ionic solution in the tracer mixing device flows out through the tracer flow-out slot, and flows into the tracer mixing device in the flow-out process; other rainwater is converged towards a road curb through a ground and enters the tracer mixing bottom plate along the curb stone for mixing; in the process, mixed rainwater enters the flushing and dissolving device; the flushing and dissolving weir body of the flushing and dissolving device is higher than the tracer mixing bottom plate; in a flowing process, a water stratification layer is formed, inorganic salt particles brought out by the rainwater of the water stratification layer are dissolved and flushed in the dissolving holes of the flushing and dissolving weir body to prevent the inorganic salt particles from flowing out and causing inaccurate testing; after the inorganic salt is dissolved in the dissolving holes, the mixed rainwater enters an urban pipeline; and
S4: after the rainwater enters the urban pipeline, a plurality of tracer detection devices are arranged in lockholes of different inspection well lids in the urban pipeline to ensure monitoring of flowing out of the rainwater; rainwater traceability monitoring is performed through the sensors of the tracer detection devices; in a monitoring process, by monitoring electric conductivity by the probes, in-water detection of the probes and monitoring of traceability substances in water are implemented; qualitative monitoring of mixed connection and semi-quantitative analysis of a mixed connection distance are implemented based on a peak value of batch conductivity variation.
